(19) United States
(12) Patent Application Publication
(10) Pub. No.: US 2008/0108661 A1
(43) Pub. Date: May 8, 2008

(54) Medicaments

(75) Inventors: Peter Cage, Loughborough (GB); Mark Furber, Loughborough (GB); Zara Khan, Macclesfield (GB); Maurice Needham, Macclesfield (GB); Peter Newham, Macclesfield (GB)

Correspondence Address: FISH & RICHARDSON P.C., P.O BOX 1022, MINNEAPOLIS, MN, 55440-1022, US

(73) Assignee: AstraZeneca AB

(21) Appl. No.: 11/866,611

(22) Filed: Oct. 3, 2007

Related U.S. Application Data
(63) Continuation-in-part of application No. PCT/SE2007/000321, filed on Apr. 4, 2007.
(60) Provisional application No. 60/790,303, filed on Apr. 6, 2006.

(51) Int. Cl. A61K 31/454; C07D 401/12; A61P 19/02; A61P 19/10

(52) U.S. Cl. 514/326; 546/209

(57) ABSTRACT

Use of a compound of formula (I): 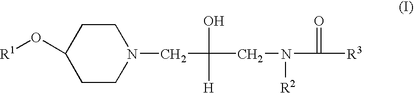
wherein: R¹ is phenyl optionally substituted by halogen, cyano, C₁₋₄ alkyl or C₁₋₄ haloalkyl; R² is hydrogen, C₁₋₆ alkyl or C₃₋₆ cycloalkyl; and R³ is a group having an NH or OH that has a calculated or measured pKa of 1.0 to 8.0; or a pharmaceutically acceptable salt thereof, in the manufacture of a medicament for use in the treatment of osteoarthritis or osteoarthrosis. The invention also concerns the benzenesulfonate salt of N-{3-[4-(3,4-dichlorophenoxy)piperidin-1-yl]-2-hydroxypropyl}-2,3-dihydro-2-oxo-4-(trifluoromethyl)-5-thiazolecarboxamide, a process for making it, and its use in therapy (for example in treating CCR3 mediated disease states).

This application is a continuation-in-part and pursuant to 35 U.S.C. § 120, claims the benefit of prior PCT International Application No. PCT/SE2007/000321, filed Apr. 4, 2007, which claims priority to U.S. Provisional Application 60/790,303, filed Apr. 6, 2006. Each of these applications is incorporated by reference in its entirety.

The present invention concerns piperidine derivatives having pharmaceutical activity as modulators of chemokine receptor (for example CCR3) activity and H₁ antagonist activity in the treatment of osteoarthritis or osteoarthrosis. The invention also concerns a particular salt of a specific piperidine derivative.

Histamine is a basic amine, 2-(4-imidazolyl)-ethylamine, and is formed from histidine by histidine decarboxylase. It is found in most tissues of the body, but is present in high concentrations in the lung, skin and in the gastrointestinal tract. At the cellular level inflammatory cells such as mast cells and basophils store large amounts of histamine. It is recognised that the degranulation of mast cells and basophils and the subsequent release of histamine is a fundamental mechanism responsible for the clinical manifestation of an allergic process. Histamine produces its actions by an effect on specific histamine G—protein coupled receptors, which are of four main types, H1, H2 H3 and H4. Histamine H₁ antagonists comprise the largest class of medications used in the treatment of patients with allergic disorders, for example rhinitis and urticaria. Antagonists of H1 are useful in controlling the allergic response by for example blocking the action of histamine on post-capillary venule smooth muscle, resulting in decreased vascular permeability, exudation and oedema. The antagonists also produce blockade of the actions of histamine on the H1 receptors on c-type nociceptive nerve fibres, resulting in decreased itching and sneezing.

Chemokines are chemotactic cytokines that are released by a wide variety of cells to attract macrophages, T cells, eosinophils, basophils and neutrophils to sites of inflammation and also play a role in the maturation of cells of the immune system. Chemokines play an important role in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C-X-C, or a) and Cys-Cys (C-C, or β) families. These are distinguished on the basis of a single amino acid insertion between the NH-proximal pair of cysteine residues and sequence similarity.

The C-X-C chemokines include several potent chemoattractants and activators of neutrophils such as interleukin-8 (IL-8) and neutrophil-activating peptide 2 (NAP-2).

The C-C chemokines include potent chemoattractants of monocytes and lymphocytes, but not neutrophils, such as human monocyte chemotactic proteins 1-3 (MCP-1, MCP-2 and MCP-3), RANTES (Regulated on Activation, Normal T Expressed and Secreted), eotaxins and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4.

Pharmaceutically active N-(2-hydroxyprop-1-yl)piperidine derivatives having is chemokine receptor modulatory activity and H1 antagonist activity are disclosed in WO2005/073192.

Osteoarthritis (OA) is a group of chronic, painful, disabling conditions affecting synovial joints and is characterized by the degeneration of matrix components of articular cartilage accompanied by the production of proinflammatory cytokines (Pelletier et al., Rheum Dis Clin North Am 19 (1993), pp. 545-568). Interleukin-1 (IL-1) beta is widely accepted as one of the proinflammatory cytokines that plays a role in the pathophysiology of OA (Dinarello, Interleukin-1, Ann N Y Acad Sci 546 (1988), pp. 122-132). The catabolic events occurring in chondrocytes include the up-regulation in genes of matrix metalloproteinases (MMPs), inducible nitric oxide synthase (iNOS) (Stadler et al., J Immunol 147 (1991), pp. 3915-3920), cyclooxygenase-2 (COX-2) (Morisset et al., J Rheumatol 25 (1998), pp. 1146-1153) and microsomal prostaglandin E synthase-1 (mPGEs 1) and the release of nitric oxide (NO) and prostaglandin E₂ (PGE₂)(Stichtenoth et al., J Immunol 167 (2001), pp. 469-474). A concommitant retardation in the anabolic activities of the chondrocytes leads to a decline in proteoglycan and collagen synthesis (Benton and Tyler, Biochem Biophys Res Commun 154 (1988), pp. 421-428, and Coll Relat Res 8 (1988), pp. 393-405).

Recent studies have shown that a number of G-Protein coupled receptors (GPCRS) and their ligands exhibit altered expression patterns in OA cartilage or in primary chondrocytes isolated from this tissue (Alaaeddine et al 2001, Arthritis Rheum. 2001, 44(7) 1633-1642, Tetlow and Woolley, Inflamm. Res 54, suppl 1 (2005) S74-S75).

The present invention is based on the finding that both the H1R and the chemokine CCR3 receptors are expressed at elevated levels in OA cartilage and their ligands are present at high levels in OA synovial fluid.

It has been found that histamine via the H1R regulates expression of PGE2, IL-6 and IL-8 in primary chondrocytes. Histamine dose-dependently induces PGE2 release from primary cultures of human chondrocyte and has been found to elevate COX-2 mRNA, and histamine and IL-1 act synergistically to regulate PGE2 expression in primary chondrocytes and in OA explant cartilage. The prevailing dogma has classified OA as a non-inflammatory joint disease. However, more recently clinical studies have demonstrated a high correlation between radiographic OA and inflammation (D'Agostino et al, Ann Rheum Dis 64 (2005), pp 1703-1709) and have linked inflammatory changes and synovitis with progression of structural changes in OA (Ayral et al, Osteoarthritis Cart 13 (2005), pp 361-367). Thus, histamine driven increases in expression of inflammatory mediators IL6, IL8 and PGE2 may play a pivotal role in OA disease progression. In addition to its role as a key mediator of inflammation, PGE2 is implicated in osteoarthritic pain via its well documented role as a sensitiser of peripheral nociceptor terminals and histamine stimulation of articular primary sensory afferents may also contribute to the mechanical hyperalgesia observed in OA.

The present inventors have also found that the CCR3 ligand, Eotaxin-2, increases expression of a number of cartilage degrading matrix metalloproteases and ADAMTS4 from human articular cartilage explants.

Accordingly it has been recognised that the inhibition of a combination of both CCR3 and H1R-mediated effects could provide a powerful therapeutic approach for treating both the signs and symptoms and disease progression in OA patients.

Accordingly, the present invention provides the use of a compound of formula (I): 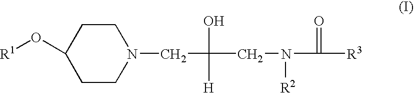
wherein:
R¹ is phenyl optionally substituted by halogen, cyano, C₁₋₄ alkyl or C₁₋₄ haloalkyl;
R² is hydrogen, C₁₋₆ alkyl or C₃₋₆ cycloalkyl; and
R³ is a group having an NH or OH that has a calculated or measured pKa of 1.0 to 8.0; or
a pharmaceutically acceptable salt thereof,
in the manufacture of a medicament for use in the treatment of osteoarthritis or osteoarthrosis.

It will be appreciated that treatment refers to both therapy or prophylaxis, unless otherwise indicated.

Compounds of formula (I) are disclosed in WO2005/073192 (published 11 Aug. 2005). pKa's of compounds of formula (I) are calculated or measured according to the methodologies described in WO2005/073192.

Certain compounds of formula (I) can exist in different isomeric forms (such as enantiomers, diastereomers, geometric isomers or tautomers). The present invention covers the use of all such isomers and mixtures thereof in all proportions.

Suitable salts include acid addition salts such as a hydrochloride, dihydrochloride, hydrobromide, phosphate, sulfate, acetate, diacetate, fumarate, maleate, tartrate, citrate, is oxalate, methanesulfonate orp-toluenesulfonate. Further examples of acid addition salts are: bisulphate, benzenesulphonate (besylate), pyruvate, succinate, ethanesulphonate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate (naphthalene-1,5-disulfonate or naphthalene-1-(sulfonic acid)-5-sulfonate), edisylate (ethane-1,2-disulfonate or ethane-1-(sulfonic acid)-2-sulfonate), isethionate (2-hydroxyethylsulfonate), 2-mesitylenesulphonate and 2-naphthalenesulphonate. Salts also include metal salts, such as an alkali metal salt (for example a sodium or potassium salt) or an alkaline earth metal salt (for example magnesium or calcium).

The compounds of formula (I) may exist as solvates (such as hydrates) and the present invention covers the use of all such solvates.

Halogen is, for example fluorine or chlorine.

Alkyl groups and moieties are straight or branched chain and are, for example, methyl, ethyl, n-propyl, iso-propyl or tert-butyl.

Cycloalkyl is monocyclic and is, for example, cyclopropyl, cyclopentyl or cyclohexyl.

Haloalkyl is an alkyl group carrying one or more (such as 1 to 6) halogen (such as chloro or fluoro atoms) and is, for example, CF₃, CH₂CF₃ or C₂F₅.

Fluoroalkyl is an alkyl group carrying one or more (such as 1 to 6) fluoro atoms and is, for example, CH₂F, CF₃, CH₂CF₃ or C₂F₅.

In one aspect the present invention provides the use of a compound of formula (I) wherein R¹ is phenyl optionally substituted by halogen, cyano or C₁₋₄ alkyl.

In another aspect the present invention the use provides a compound of formula (I) wherein R¹ is phenyl substituted with one, two or three of: halogen (such as fluoro or chloro), cyano or C₁₋₄ alkyl (such as methyl); for example R¹ is phenyl substituted by one, two or three of: fluoro, chloro, methyl or cyano. In another aspect R¹ is phenyl substituted by one, two or three (such as two or three) of: fluoro, chloro, cyano or methyl (such as chloro, cyano or methyl). R¹ is, for example, 3,4-dichlorophenyl, 2-methyl-3-chloro-4-cyanophenyl, 2-methyl-4-chlorophenyl, 3-methyl-2,4-dichlorophenyl, 2-methyl-3,4-dichlorophenyl, 3-chloro-4-cyanophenyl, 3,4-difluorophenyl, 3-fluoro-4-chlorophenyl or 4-chlorophenyl (such as 2-methyl-4-chlorophenyl, 3-methyl-2,4-dichlorophenyl, 2-methyl-3,4-dichlorophenyl, 3-chloro-4-cyanophenyl, 3,4-difluorophenyl, 3-fluoro-4-chlorophenyl or 4-chlorophenyl). In a still further aspect R¹ is 3,4-dichlorophenyl or 3-chloro-4-cyanophenyl.

In a further aspect of the invention R¹ is phenyl substituted by one or more of chloro or methyl and optionally further substituted by fluoro. For example R¹ is 2-methyl-4-chlorophenyl, 3-methyl-2,4-dichlorophenyl, 2-methyl-3,4-dichlorophenyl, 3-fluoro-4-chlorophenyl, 4-chlorophenyl or 3,4-dichlorophenyl.

In another aspect of the invention R¹ is 3,4-dichlorophenyl, 2-methyl-4-chlorophenyl, 3-methyl-2,4-dichlorophenyl, 2-methyl-3,4-dichlorophenyl or 2-methyl-3-chloro-4-cyanophenyl.

In a still further aspect the present invention provides the use of a compound of formula (I) wherein R² is hydrogen or C₁₋₄ alkyl (such as methyl).

In yet another aspect of the invention R² is hydrogen.

The acidic NH (that is the NH having a calculated or measured pKa of 1.0 to 8.0) of R³ can be part of a ring or it can be part of a substituent on an aryl or heterocyclyl ring. The acidic OH (that is the OH having a calculated or measured pKa of 1.0 to 8.0) of R³ can be a substituent or part of a substituent (such an OH in a carboxylic acid group) on an aryl or heterocyclyl ring. Thus, for example, the acidic OH of R³ can be part of an acidic phenol, in a carboxylic acid, or in a hydroxy aromatic heterocyclyl (such as a hydroxypyridine which may tautomerise to a pyridone).

Aryl includes optionally substituted phenyl and naphthyl.

Heterocyclyl is an optionally substituted aromatic or non-aromatic 5- or 6-membered ring, comprising, as required, at least one heteroatom selected from the group comprising nitrogen, oxygen and sulphur; or an N-oxide thereof, or an S-oxide or S-dioxide thereof. Heterocyclyl is, for example, furyl, thienyl (also known as thiophenyl), pyrrolyl, 2,5-dihydropyrrolyl, thiazolyl (for example in 2-oxo-2,3-dihydro-1,3-thiazolyl), isothiazolyl, pyrazolyl, oxazolyl, isoxazolyl, imidazolyl, triazolyl (for example in 1H-1,2,3-triazolyl), pyridinyl (for example in 6-oxo-1,6-dihydro-pyridinyl) or pyrimidinyl.

In an aspect of the present invention the acidic NH of R³ is part of a suitably substituted ring (for example part of a pyrrolyl, 2,5-dihydropyrrolyl, thiazolyl, isothiazolyl, pyrazolyl, oxazolyl, isoxazolyl, imidazolyl, triazolyl, pyridinyl or pyrimidinyl ring) or part of a substituent on a suitably substituted aryl (for example phenyl or naphthyl) or suitably substituted heterocyclyl (for example furyl, thienyl, pyrrolyl, 2,5-dihydropyrrolyl, thiazolyl, isothiazolyl, pyrazolyl, oxazolyl, isoxazolyl, imidazolyl, triazolyl, pyridinyl or pyrimidinyl) ring.

In another aspect of the present invention the acidic OH of R³ is a substituent or part of a substituent (such an OH in a carboxylic acid group) on a suitably substituted aryl (for example phenyl or naphthyl) or suitably substituted heterocyclyl (for example furyl, thienyl, pyrrolyl, 2,5-dihydropyrrolyl, thiazolyl, isothiazolyl, pyrazolyl, oxazolyl, isoxazolyl, imidazolyl, triazolyl, pyridinyl or pyrimidinyl) ring. Thus, for example, the acidic OH of R³ can be part of an acidic phenol (substituted or unsubstituited), in a carboxylic acid, or in a suitably substituted hydroxy aromatic heterocyclyl (such as a hydroxypyridine which may tautomerise to a pyridone). Further examples of suitably substituted hydroxy aromatic heterocyclyl are hydroxyquinolines, hydroxyisoquinolines and hydroxybenzimidazoles.

In one aspect of the present invention when the acidic NH of R³ is part of a suitably substituted ring it is, for example, part of a 2-oxo-thiazol-5-yl, 2-oxo-oxazol-5-yl, 2-oxo-imidazol-5-yl, 1H-1,2,3-triazol-4-yl, 4-oxo-1H-1,4-dihydropyridin-3-yl, 2,6-dioxo-1H-1,2,3,6-tetrahydropyrimidin-4-yl, 6-oxo-1H-1,6-dihydropyridin-3-yl or 2H-tetrazol-5-yl ring.

In another aspect of the present invention when the acidic NH of R³ is part of a suitably substituted ring it is, for example, part of a 2-oxo-thiazol-5-yl, 1H-1,2,3-triazol-4-yl or 6-oxo-1H-1,6-dihydropyridin-3-yl ring.

In a further aspect of the present invention when the acidic NH of R³ is part of a substituent it is, for example, part of NHS(O)₂(C₁₋₄ alkyl).

In another aspect the present invention provides the use of a compound of formula (I) wherein R³ is a group having an NH or OH that has a calculated or measured pKa of 3 to 6.5.

In yet another aspect the present invention provides the use of a compound of formula (I) wherein R³ is a group having an NH or OH that has a calculated or measured pKa of 1.0 to 8.0 (for example 3 to 6.5), the group R³ being, for example,
2-oxo-thiazol-5-yl having a suitable electron withdrawing substituent {such as C₁₋₄ fluoroalkyl (for example CF₃, CH₂ CF₃ or C₂F₅), an aryl group (for example 4-fluorophenyl), a heterocyclyl group (for example pyridyl) or a group CH₂S(O)₂(C₁₋₄ alkyl)} in the 4-position;
2-oxo-oxazol-5-yl having a suitable electron withdrawing substituent {such as C₁₋₄ fluoroalkyl (for example CF₃, CH₂CF₃ or C₂F₅) or CH₂S(O)₂(C₁₋₄ alkyl)} in the 4-position;
1H-1,2,3-triazol-4-yl having a suitable substituent {such as C₁₋₄ alkyl (for example CH₃ or CH(CH₃)₂), C₃₋₆ cycloalkyl (for example cyclopropyl), C₁₋₄ fluoroalkyl (for example CF₃, CH₂CF₃ or C₂F₅), S—R⁴ (wherein R⁴ is C₁₋₄ alkyl [for example CH₃], C₁₋₄ fluoroalkyl [for example CF₃, CH₂CF₃ or C₂F₅] or C₃₋₆ cycloalkyl [for example cyclopropyl]), NHS(O)₂(C₁₋₄ alkyl), N(C₁₋₄ alkyl)S(O)₂(C₁₋₄ alkyl), an aryl group (for example 4-fluorophenyl), a heterocyclyl group (for example pyridyl) or a group CH₂S(O)₂(C₁₋₄ alkyl)} in the 5-position;
4-oxo-1H-1,4-dihydropyridin-3-yl having a suitable electron withdrawing substituent {such as C₁₋₄ fluoroalkyl (for example CF₃ or C₂F₅)} in the 2-position;
2,6-dioxo-1H-1,2,3,6-tetrahydropyrimidin-4-yl having a suitable substituent {such as C₁₋₄ alkyl (for example CH₃), C₃₋₆ cycloalkyl (for example cyclopropyl) or CH₂(C₁₋₃ fluoroalkyl) (for example CH₂CF₃)} in the 3-position and optionally substituted in one or more other ring positions;
6-oxo-1H-1,6-dihydropyridin-3-yl having a suitable electron withdrawing substituent {such as C₁₋₄ fluoroalkyl (for example CF₃, CH₂CF₃ or C₂F₅), cyano or phenyl} in the 2-position and/or the 5-position and optionally substituted in one or more other ring positions;
6-oxo-1H-1,6-dihydropyridin-3-yl having CH₂CO₂H on the ring nitrogen and optionally substituted in one or more other ring positions;
2H-tetrazol-5-yl;
a CO₂H, CH₂CO₂H or OCH₂CO₂H group on an optionally substituted phenyl, optionally substituted CH₂O phenyl, optionally substituted naphthyl ring or optionally substituted acylated (such as with C(O)(C₁₋₄ alkyl)) dihydroisoquinolinyl ring; or,
an NHS(O)₂(C₁₋₄ alkyl) (for example NHS(O)₂CH₃) group on an optionally substituted aromatic heterocyclyl ring (for example pyridinyl, pyrimidinyl or thiazolyl);
   or, where possible, a tautomer thereof.

In one aspect of the invention acylated (such as with C(O)(C₁₋₄ alkyl)) dihydroisoquinolinyl carries the CO₂H, CH₂CO₂H or OCH₂CO₂H group on position 7.

In yet another aspect the present invention provides the use of a compound of formula (I) wherein R³ is a group having an NH or OH that has a calculated or measured pKa of 1.0 to 8.0 (for example 3 to 6.5), the group R³ being, for example,
2-oxo-thiazol-5-yl having a suitable electron withdrawing substituent {such as C₁₋₄ fluoroalkyl (for example CF₃, CH₂CF₃ or C₂F₅), an aryl group (for example 4-fluorophenyl), a heterocyclyl group (for example pyridyl) or a group CH₂S(O)₂(C₁₋₄ alkyl)} in the 4-position;
2-oxo-oxazol-5-yl having a suitable electron withdrawing substituent {such as C₁₋₄ fluoroalkyl (for example CF₃, CH₂CF₃ or C₂F₅) or CH₂S(O)₂(C₁₋₄ alkyl)} in the 4-position;
1H-1,2,3-triazol-4-yl having a suitable substituent {such as C₁₋₄ alkyl (for example CH₃), C₃₋₆ cycloalkyl (for example cyclopropyl), C₁₋₄ fluoroalkyl (for example CF₃, CH₂CF₃ or C₂F₅), S—R⁴ (wherein R⁴ is C₁₋₄ alkyl [for example CH₃], C₁₋₄ fluoroalkyl [for example CF₃, CH₂CF₃ or C₂F₅] or C₃₋₆ cycloalkyl [for example cyclopropyl]), NHS(O)₂(C₁₋₄ alkyl), an aryl group (for example 4-fluorophenyl), a heterocyclyl group (for example pyridyl) or a group CH₂S(O)₂(C₁₋₄ alkyl)} in the 5-position;
4-oxo-1H-1,4-dihydropyridin-3-yl having a suitable electron withdrawing substituent {such as C₁₋₄ fluoroalkyl (for example CF₃ of C₂F₅)} in the 2-position;
2,6-dioxo-1H-1,2,3,6-tetrahydropyrimidin-4-yl having a suitable substituent {such as C₁₋₄ alkyl (for example CH₃), C₃₋₆ cycloalkyl (for example cyclopropyl) or CH₂(C₁₋₃ fluoroalkyl) (for example CH₂CF₃)} in the 3-position;
6-oxo-1H-1,6-dihydropyridin-3-yl having a suitable electron withdrawing substituent {such as C₁₋₄ fluoroalkyl (for example CF₃, CH₂CF₃ or C₂F₅) or cyano} in the 2-position or the 5-position and optionally substituted in other positions;
2H-tetrazol-5-yl;
a CO₂H group on an optionally substituted phenyl or naphthyl ring; or,
an NHS(O)₂(C₁₋₄ alkyl) (for example NHS(O)₂CH₃) group on an optionally substituted aromatic heterocyclyl ring (for example pyridinyl, pyrimidinyl or thiazolyl);
   or, where possible, a tautomer thereof.

Where indicated above that a heterocyclyl ring in R³ may be optionally substituted it can be optionally substituted by, for example: fluoro, chloro, bromo, C₁₋₄ alkyl (for example methyl), C₃₋₆ cycloalkyl (for example cyclopropyl), C₁₋₄ fluoroalkyl (for example CF₃, CH₂CF₃ or C₂F₅), S—R⁴ (wherein R⁴ is C₁₋₄ alkyl [for example CH₃], C₁₋₄ fluoroalkyl [for example CF₃, CH₂CF₃ or C₂F₅] or C₃₋₆ cycloalkyl [for example cyclopropyl]), cyano, S(O)₂(C₁₋₄ alkyl) (for example S(O)₂CH₃) or S(O)₂NH(C₁₋₄ alkyl) (for example S(O)₂NHCH₃).

Where indicated above that a phenyl or naphthyl ring in R³ may be optionally substituted it can be optionally substituted by, for example, halogen, cyano, C₁₋₄ alkyl, C₁₋₄ alkoxy, C₁₋₄ fluoroalkyl (for example CF₃, CH₂CF₃ or C₂F₅)}, OCF₃, SCF₃, nitro, S(C₁₋₄ alkyl), S(O)(C₁₋₄ alkyl), S(O)₂(C₁₋₄ alkyl), S(O)₂NH(C₁₋₄ alkyl), S(O)₂N(C₁₋₄ alkyl)₂, NHC(O)(C₁₋₄ alkyl), NHS(O)₂(C₁₋₄ alkyl).

In one aspect of the invention R³ is:
2-oxo-thiazol-5-yl having C₁₋₄ fluoroalkyl (for example CF₃, CH₂CF₃ or C₂F₅) in the 4-position;
1H-1,2,3-triazol-4-yl having a suitable substituent {such as C₁₋₄ alkyl (for example CH₃) or S—R⁴ (wherein R⁴ is C₁₋₄ fluoroalkyl [for example CF₃, CH₂CF₃ or C₂F₅])} in the 5-position;
2,6-dioxo-1H-1,2,3,6-tetrahydropyrimidin-4-yl having a suitable substituent {such as C₁₋₄ alkyl (for example CH₃) or C₁₋₄ fluoroalkyl (for example CF₃, CH₂CF₃ or C₂F₅)} in the 3-position;
6-oxo-1H-1,6-dihydropyridin-3-yl having a suitable electron withdrawing substituent {such as C₁₋₄ fluoroalkyl (for example CF₃, CH₂CF₃ or C₂F₅) or cyano} in the 2-position or the 5-position and optionally substituted in other positions;
a CO₂H group on an optionally substituted naphthyl ring; or,
an NHS(O)₂(C₁₋₄ alkyl) (for example NHS(O)₂CH₃) group on an optionally substituted aromatic heterocyclyl ring (for example pyridinyl, pyrimidinyl or thiazolyl);
   or, where possible, a tautomer thereof, the optional substituents being as defined above.

In yet another aspect the present invention provides the use of a compound of formula (I) wherein R³ is:
2-oxo-thiazol-5-yl having a suitable electron withdrawing substituent {such as C₁₋₄ fluoroalkyl (for example CF₃, CH₂CF₃ or C₂F₅), a phenyl group (for example 4-fluorophenyl) or a heterocyclyl group (for example pyridyl)} in the 4-position;
1H-1,2,3-triazol-4-yl having a suitable substituent {such as C₁₋₄ alkyl (for example CH₃ or CH(CH₃)₂), C₁₋₄ fluoroalkyl (for example CF₃, CH₂CF₃ or C₂F₅), S—R⁴ (wherein R⁴ is C₁₋₄ alkyl [for example CH₃] or C₁₋₄ fluoroalkyl [for example CF₃, CH₂CF₃ or C₂F₅]), N(C₁₋₄ alkyl)S(O)₂(C₁₋₄ alkyl) or a phenyl group (for example 4-fluorophenyl)} in the 5-position; or,
6-oxo-1H-1,6-dihydropyridin-3-yl having C₁₋₄ fluoroalkyl (for example CF₃, CH₂CF₃ or C₂F₅) or cyano in the 2-position or the 5-position.

In another aspect the present invention provides a compound of formula (I) wherein R³ is:
2-oxo-thiazol-5-yl having CF₃ or C₂F₅ in the 4-position;
1H-1,2,3-triazol-4-yl having CF₃, C₂F₅, SCF₃, SCH₂CF₃ or SC₂F₅ (for example CF₃ or SCH₂CF₃) in the 5-position; or,
6-oxo-1H-1,6-dihydropyridin-3-yl having CF₃ or C₂F₅ in the 2-position.

In yet another aspect the present invention provides the use of a compound of formula (I) wherein the 2-hydroxy group has the stereochemistry shown below:

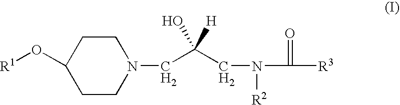

In a further aspect the present invention provides the benzenesulfonate salt of N-{3-[4-(3,4-dichlorophenoxy)piperidin-1-yl]-2-hydroxypropyl}-2,3-dihydro-2-oxo-4-(trifluoromethyl)-5-thiazolecarboxamide (which is an example of pharmaceutically acceptable salt form of a compound of formula (I)). In a still further aspect the present invention provides the benzenesulfonate salt of N-{(2R)-3-[4-(3,4-dichlorophenoxy)piperidin-1-yl]-2-hydroxypropyl}-2,3-dihydro-2-oxo-4-(trifluoromethyl)-5-thiazolecarboxamide.

In some embodiments, a compound described herein can be enriched for a particular stereoisomer, e.g., the R isomer. For example N-{3-[4-(3,4-dichlorophenoxy)piperidin-1-yl]-2-hydroxypropyl}-2,3-dihydro-2-oxo-4-(trifluoromethyl)-5-thiazolecarboxamide can be resolved to a high enantiomeric excess (e.g., 60%, 70%, 80%, 85%, 90%, 95%, 99% or greater). In some embodiments, a compound is a substantially pure stereoisomer. For example, the compound is substantially a single R isomer, substantially free of other isomers such as the S isomer, (such as 75% free, for example 85% free, such as 95% free, for example 99% free).

In another aspect the present invention provides N-{(2R)-3-[4-(3,4-dichlorophenoxy)piperidin-1-yl]-2-hydroxypropyl}-2,3-dihydro-2-oxo-4-(trifluoromethyl)-5-thiazolecarboxamide benzenesulfonate Form A having an X-ray powder diffraction pattern containing specific peaks at: 4.4 (±0.1°), 8.7 (±0.1°), 11.6 (±0.1°), 17.5 (±0.1°), 20.2 (±0.1°) and 21.9 (±0.1°) 2θ.

In yet another aspect the present invention provides N-{(2R)-3-[4-(3,4-dichlorophenoxy)piperidin-1-yl]-2-hydroxypropyl}-2,3-dihydro-2-oxo-4-(trifluoromethyl)-5-thiazolecarboxamide benzenesulfonate Form B having an X-ray powder diffraction pattern containing specific peaks at: 4.1 (±0.1°), 8.2 (±0.1°), 16.4 (±0.1°), 20.5 (±0.1°), 22.9 (±0.1°) and 24.7 (±0.1°) 2θ.

In a still further aspect the present invention provides a process for the preparation of the benzenesulfonate salt of N-{3-[4-(3,4-dichlorophenoxy)piperidin-1-yl]-2-hydroxypropyl}-2,3-dihydro-2-oxo-4-(trifluoromethyl)-5-thiazolecarboxamide comprising treating N-{3-[4-(3,4-dichlorophenoxy)piperidin-1-yl]-2-hydroxypropyl}-2,3-dihydro-2-oxo-4-(trifluoromethyl)-5-thiazolecarboxamide with benzenesulfonic acid in a suitable solvent (such as an aliphatic alcohol, for example methanol) at ambient temperature (for example 0-35° C.).

Compounds of formula (I) can be prepared by methods described, or analogous to those described, in WO 2005/073192. Intermediates for such processes can be prepared by methods described, or analogous to those described in WO 2005/073192.

In a further aspect the invention provides a compound for use according to the invention, the compound being:
N-{(2R)-3-[4-(3,4-Dichlorophenoxy)piperidin-1-yl]-2-hydroxypropyl}-6-oxo-2-(trifluoromethyl)-1,6-dihydropyridine-3-carboxamide;
N-{(2R)-3-[4-(2,4-Dichloro-3-methylphenoxy)piperidin-1-yl]-2-hydroxypropyl}-6-oxo-2-(trifluoromethyl)-1,6-dihydropyridine-3-carboxamide;
5-Bromo-N-{(2R)-3-[4-(3,4-dichlorophenoxy)piperidin-1-yl]-2-hydroxypropyl}-6-oxo-2-(trifluoromethyl)-1,6-dihydropyridine-3-carboxamide;
N-{(2R)-3-[4-(3,4-Dichlorophenoxy)piperidin-1-yl]-2-hydroxypropyl}-2,3-dihydro-2-oxo-4-(trifluoromethyl)-5-thiazolecarboxamide;
N-{(2S)-3-[4-(3,4-Dichlorophenoxy)piperidin-1-yl]-2-hydroxypropyl}-N-methyl-2-oxo-4-(trifluoromethyl)-2,3-dihydro-1,3-thiazole-5-carboxamide;
N-{(2S)-3-[4-(2,4-Dichloro-3-methylphenoxy)piperidin-1-yl]-2-hydroxypropyl}-N-methyl-2-oxo-4-(trifluoromethyl)-2,3-dihydro-1,3-thiazole-5-carboxamide;
N-{(2R)-3-[4-(3,4-Dichlorophenoxy)piperidin-1-yl]-2-hydroxypropyl}-2-oxo-4-(pentafluoroethyl)-2,3-dihydro-1,3-thiazole-5-carboxamide;
N-{(2R)-3-˜4-(3,4-Dichlorophenoxy)piperidin-1-yl]-2-hydroxypropyl}-5-methyl-1H-1,2,3-triazole-4-carboxamide;
N-{(2R)-3-[4-(2,4-Dichloro-3-methylphenoxy)piperidin-1-yl]-2-hydroxypropyl}-5-methyl-1H-1,2,3-triazole-4-carboxamide;
5-Cyano-N-{(2R)-3-[4-(3,4-dichlorophenoxy)piperidin-1-yl]-2-hydroxypropyl}-6-oxo-2-(trifluoromethyl)-1,6-dihydropyridine-3-carboxamide;
5-Cyano-N-{(2R)-3-[4-(2,4-dichloro-3-methylphenoxy)piperidin-1-yl]-2-hydroxypropyl}-6-oxo-2-(trifluoromethyl)-1,6-dihydropyridine-3-carboxamide;
5-Cyano-N-{(2R)-3-[4-(3,4-dichlorophenoxy)piperidin-1-yl]-2-hydroxypropyl}-6-oxo-2-phenyl-1,6-dihydropyridine-3-carboxamide;
5-Cyano-N-{(2R)-3-[4-(2,4-dichloro-3-methylphenoxy)piperidin-1-yl]-2-hydroxypropyl}-6-oxo-2-phenyl-1,6-dihydropyridine-3-carboxamide;
5-Cyano-N-{(2R)-3-[4-(3,4-dichlorophenoxy)piperidin-1-yl]-2-hydroxypropyl}-6-oxo-2-(trifluoromethyl)-1,6-dihydropyridine-3-carboxamide;
N-{(2R)-3-[4-(3,4-Dichlorophenoxy)piperidin-1-yl]-2-hydroxypropyl}-2,6-dioxo-3-(2,2,2-trifluoroethyl)-1,2,3,6-tetrahydropyrimidine-4-carboxamide;
5-Cyano-2-cyclopropyl-N-[(2R)-3-[4-(3,4-dichlorophenoxy)-1-piperidinyl]-2-hydroxypropyl]-1,6-dihydro-6-oxo-3-pyridinecarboxamide;
5-Cyano-2-cyclopropyl-N-[(2R)-3-[4-(2,4-dichloro-3-methylphenoxy)-1-piperidinyl]-2-hydroxypropyl]-1,6-dihydro-6-oxo-3-pyridinecarboxamide;
N-{(2R)-3-[4-(3,4-Dichlorophenoxy)piperidin-1-yl]-2-hydroxypropyl}-6-[(methylsulfonyl)amino]-4-(trifluoromethyl)nicotinamide;
N-{(2R)-3-[4-(3,4-Dichlorophenoxy)piperidin-1-yl]-2-hydroxypropyl}-5-[(2,2,2-trifluoroethyl)thio]-1H-1,2,3-triazole-4-carboxamide;
4-[({(2R)-3-[4-(3,4-Dichlorophenoxy)piperidin-1-yl]-2-hydroxypropyl}amino)-carbonyl]-1-naphthoic acid;
N-{(2R)-3-[4-(3,4-Dichlorophenoxy)piperidin-1-yl]-2-hydroxypropyl}-2-[(methylsulfonyl)amino]-4-(trifluoromethyl)-1,3-thiazole-5-carboxamide;
N-{(2R)-3-[4-(4-Chloro-2-methylphenoxy)piperidin-1-yl]-2-hydroxypropyl}-2-oxo-4-(trifluoromethyl)-2,3-dihydro-1,3-thiazole-5-carboxamide;
[5-[({(2R)-3-[4-(3,4-Dichlorophenoxy)piperidin-1-yl]-2-hydroxypropyl}amino)-carbonyl]-2-oxo-4-(trifluoromethyl)pyridin-1(2H)-yl]acetic acid;
N-{(2R)-3-[4-(3,4-Dichloro-2-methylphenoxy)piperidin-1-yl]-2-hydroxypropyl}-2-oxo-4-(trifluoromethyl)-2,3-dihydro-1,3-thiazole-5-carboxamide;
N-{(2R)-3-[4-(3,4-Dichlorophenoxy)piperidin-1-yl]-2-hydroxypropyl}-4-(4-fluorophenyl)-2-oxo-2,3-dihydro-1,3-thiazole-5-carboxamide;
N-{(2R)-3-[4-(3,4-Dichlorophenoxy)piperidin-1-yl]-2-hydroxypropyl}-5-(4-fluorophenyl)-1H-1,2,3-triazole-4-carboxamide;
N-{(2R)-3-[4-(3-Chloro-4-cyanophenoxy)piperidin-1-yl]-2-hydroxypropyl}-2-oxo-4-(trifluoromethyl)-2,3-dihydro-1,3-thiazole-5-carboxamide;
N-{(2S)-3-[4-(3,4-Dichlorophenoxy)piperidin-1-yl]-2-hydroxypropyl}-2-oxo-4-(trifluoromethyl)-2,3-dihydro-1,3-thiazole-5-carboxamide;
N-{(2S)-3-[4-(3-Chloro-4-cyanophenoxy)piperidin-1-yl]-2-hydroxypropyl}-N-methyl-2-oxo-4-(trifluoromethyl)-2,3-dihydro-1,3-thiazole-5-carboxamide;
N-{(2R)-3-[4-(2,4-Dichloro-3-methylphenoxy)piperidin-1-yl]-2-hydroxypropyl}-2-oxo-4-(trifluoromethyl)-2,3-dihydro-1,3-thiazole-5-carboxamide;
N-{(2R)-3-[4-(3-Chloro-4-cyanophenoxy)piperidin-1-yl]-2-hydroxypropyl}-5-isopropyl-1H-1,2,3-triazole-4-carboxamide;
N-{(2S)-3-[4-(3-Chloro-4-cyanophenoxy)piperidin-1-yl]-2-hydroxypropyl}-5-isopropyl-N-methyl-1H-1,2,3-triazole-4-carboxamide;
N-{(2R)-3-[4-(3,4-Dichlorophenoxy)piperidin-1-yl]-2-hydroxypropyl}-2-oxo-4-(2,2,2-trifluoroethyl)-2,3-dihydro-1,3-thiazole-5-carboxamide;
N-{(2R)-3-[4-(3,4-Dichlorophenoxy)piperidin-1-yl]-2-hydroxypropyl}-2-oxo-4-pyridin-2-yl-2,3-dihydro-1,3-thiazole-5-carboxamide;
N-{(2R)-3-[4-(3,4-Dichlorophenoxy)piperidin-1-yl]-2-hydroxypropyl}-6-oxo-2-(pentafluoroethyl)-1,6-dihydropyridine-3-carboxamide;
N-{(2R)-3-[4-(3,4-Dichlorophenoxy)piperidin-1-yl]-2-hydroxypropyl}-5-(methylthio)-1H-1,2,3-triazole-4-carboxamide;
N-{(2R)-3-[4-(3,4-Dichlorophenoxy)piperidin-1-yl]-2-hydroxypropyl}-2-oxo-4-(trifluoromethyl)-2,3-dihydro-1,3-oxazole-5-carboxamide;
N-{(2R)-3-[4-(3,4-Dichlorophenoxy)piperidin-1-yl]-2-hydroxypropyl}-5-(trifluoromethyl)-1H-1,2,3-triazole-4-carboxamide;
N-{(2R)-3-[4-(3,4-Dichlorophenoxy)piperidin-1-yl]-2-hydroxypropyl}-5-[methyl(methylsulfonyl)amino]-1H-1,2,3-triazole-4-carboxamide;
N-{(2R)-3-[4-(3-Chloro-4-cyano-2-methylphenoxy)piperidin-1-yl]-2-hydroxypropyl}-2-oxo-4-(trifluoromethyl)-2,3-dihydro-1,3-thiazole-5-carboxamide;
N-{(2R)-3-[4-(3-Chloro-4-cyanophenoxy)piperidin-1-yl]-2-hydroxypropyl}-5-(trifluoromethyl)-1H-1,2,3-triazole-4-carboxamide;
2-Chloro-5-[({(2R)-3-[4-(3,4-dichloro-2-methylphenoxy)piperidin-1-yl]-2-hydroxypropyl}amino)carbonyl]benzoic acid;
4-Chloro-3-[({(2R)-3-[4-(3,4-dichloro-2-methylphenoxy)piperidin-1-yl]-2-hydroxypropyl}amino)carbonyl]benzoic acid;
4-Chloro-3-[2-({(2R)-3-[4-(3,4-dichloro-2-methylphenoxy)piperidin-1-yl]-2-hydroxypropyl}amino)-2-oxoethoxy]benzoic acid;
{2-Chloro-5-[({(2R)-3-[4-(3,4-dichloro-2-methylphenoxy)piperidin-1-yl]-2-hydroxypropyl}amino)carbonyl]phenoxy}acetic acid;
3-[2-({(2R)-3-[4-(3,4-Dichloro-2-methylphenoxy)piperidin-1-yl]-2-hydroxypropyl}amino)-2-oxoethoxy]benzoic acid; or,
{3-[({(2R)-3-[4-(3,4-Dichloro-2-methylphenoxy)piperidin-1-yl]-2-hydroxypropyl}amino)carbonyl]phenoxy}acetic acid;
   or a pharmaceutically acceptable salt thereof.

In a further aspect the invention provides the use of N-{(2R)-3-[4-(3,4-dichlorophenoxy)piperidin-1-yl]-2-hydroxypropyl}-2,3-dihydro-2-oxo-4-(trifluoromethyl)-5-thiazolecarboxamide, or a pharmaceutically acceptable salt thereof (for example a benzenesulfonate salt), in the manufacture of a medicament for the treatment of osteoarthritis or osteoarthrosis.

The invention also provides the benzenesulfonate salt of N-{3-[4-(3,4-dichlorophenoxy)piperidin-1-yl]-2-hydroxypropyl}-2,3-dihydro-2-oxo-4-(trifluoromethyl)-5-thiazolecarboxamide (for example its (2R) enantiomer) for use in therapy or prophylaxis.

The invention further provides the use of the benzenesulfonate salt of N-{3-[4-(3,4-dichlorophenoxy)piperidin-1-yl]-2-hydroxypropyl}-2,3-dihydro-2-oxo-4-(trifluoromethyl)-5-thiazolecarboxamide (for example its (2R) enantiomer), in the manufacture of a medicament for use in the treatment of a CCR3 mediated disease state (such as:
(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis; sarcoidosis; farmer's lung and related diseases; nasal polyposis; fibroid lung, idiopathic interstitial pneumonia, antitussive activity, treatment of chronic cough associated with inflammatory conditions of the airways or iatrogenic induced cough;
(2) (bone and joints) rheumatic, infectious or autoimmune arthrides; seronegative spondyloarthropathies (such as ankylosing spondylitis, psoriatic arthritis or Reiter's disease); Behçet's disease; Sjogren's syndrome; or systemic sclerosis; or,
(3) (skin and eyes) psoriasis, atopic dermatitis, contact dermatitis or other eczmatous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia greata or vernal conjunctivitis;
   in a warm blooded animal, such as man).

In another aspect the present invention provides a method of treating a CCR3 mediated disease state which comprises administering to a patient a therapeutically effective amount of the benzenesulfonate salt of N-{3-[4-(3,4-dichlorophenoxy)piperidin-1-yl]-2-hydroxypropyl}-2,3-dihydro-2-oxo-4-(trifluoromethyl)-5-thiazolecarboxamide (for example its (2R) enantiomer).

In a further aspect the benzenesulfonate salt of N-{3-[4-(3,4-dichlorophenoxy)piperidin-1-yl]-2-hydroxypropyl}-2,3-dihydro-2-oxo-4-(trifluoromethyl)-5-thiazolecarboxamide (for example its (2R) enantiomer) is useful in the treatment of asthma {such as bronchial, allergic, intrinsic, extrinsic or dust asthma, particularly chronic or inveterate asthma (for example late asthma or airways hyper-responsiveness)}; or rhinitis {including acute, allergic, atrophic or chronic rhinitis, such as rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis}.

In a still further aspect the benzenesulfonate salt of N-{3-[4-(3,4-dichlorophenoxy)piperidin-1-yl]-2-hydroxypropyl}-2,3-dihydro-2-oxo-4-(trifluoromethyl)-5-thiazolecarboxamide (for example its (2R) enantiomer) is useful in the treatment of asthma.

The present invention also provides the use of the benzenesulfonate salt of N-{3-[4-(3,4-dichlorophenoxy)piperidin-1-yl]-2-hydroxypropyl}-2,3-dihydro-2-oxo-4-(trifluoromethyl)-5-thiazolecarboxamide (for example its (2R) enantiomer) in the manufacture of a medicament for use in the treatment of asthma or rhinitis.

Compounds of formula (I) and their pharmaceutically acceptable salts have activity as modulators of chemokine receptor (for example CCR3) activity and are also H₁ antagonists, and may be used in the treatment of arthritides associated with osteoarthritis or osteoarthrosis, both primary and secondary to, for example, congenital hip dysplasia.

According to a further feature of the invention there is provided a compound of formula (I), or a pharmaceutically acceptable salt thereof, for use in a method of treatment of osteoarthritis or osteoarthrosis of a warm blooded animal (such as man).

In another aspect of the present invention there is provided a method for treating osteoaerthritis or osteoarthrosis, in a warm blooded animal, such as man, in need of such treatment, which comprises administering to said animal an effective amount of a compound of the formula (I), or a pharmaceutically acceptable salt thereof.

In order to use a compound of formula (I), or a pharmaceutically acceptable salt thereof, (active ingredient) for the therapeutic treatment of a warm blooded animal, such as man, said ingredient is normally formulated in accordance with standard pharmaceutical practice as a pharmaceutical composition.

The present invention also provides a composition comprising the benzenesulfonate salt of N-{3-[4-(3,4-dichlorophenoxy)piperidin-1-yl]-2-hydroxypropyl}-2,3-dihydro-2-oxo-4-(trifluoromethyl)-5-thiazolecarboxamide (for example its (2R) enantiomer) in admixture with a carrier, diluent or adjuvant.

Depending on the mode of administration, the pharmaceutical composition will, for example, comprise from 0.05 to 99% w (percent by weight), such as from 0.05 to 80% w, for example from 0.10 to 70% w, such as from 0.10 to 50% w, of active ingredient, all percentages by weight being based on total composition.

A pharmaceutical composition can be administered in a standard manner for the disease condition that it is desired to treat, for example by topical (such as to the lung and/or airways or to the skin), oral, rectal or parenteral administration (for example, intra-articular). For these purposes the compounds of formula (I) may be formulated by means known in the art into the form of, for example, aerosols, dry powder formulations, tablets, capsules, syrups, powders, granules, aqueous or oily solutions or suspensions, (lipid) emulsions, dispersible powders, suppositories, ointments, creams, drops and sterile injectable aqueous or oily solutions or suspensions.

A suitable pharmaceutical composition is one suitable for oral administration in unit dosage form, for example a tablet or capsule which contains between 0.1 mg and 1 g of active ingredient.

Alternatively, a pharmaceutical composition is one suitable for intravenous, subcutaneous or intramuscular injection. Alternatively, a pharmaceutical composition is one suitable for intra-articular administration.

Each patient may receive, for example, an intra-articular, intravenous, subcutaneous or intramuscular dose of 0.01 mgkg⁻¹ to 100 mgkg⁻¹ of the compound, for example in the range of 0.1 mgkg⁻¹ to 20 mgkg⁻¹ of this invention, the composition being administered 1 to 4 times per day. The intra-articular, intravenous, subcutaneous or intramuscular dose may be given by means of a bolus injection. Alternatively, the intra-articular or intravenous dose may be given by continuous infusion over a period of time. Alternatively each patient can receive a daily oral dose which is approximately equivalent to the daily parenteral dose, the composition being administered 1 to 4 times per day.

The invention further relates to combination therapies or compositions wherein a compound of formula (I), or a pharmaceutically acceptable salt thereof, or a pharmaceutical composition comprising a compound of formula (I), or a pharmaceutically acceptable salt thereof, is administered concurrently (possibly in the same composition) or sequentially with an agent for the treatment of osteoarthritis or osteoarthrosis.

In particular a compound of formula (I) can be combined with a histamine type 1 receptor antagonist such as cetirizine, loratadine, desloratadine, fexofenadine, astemizole, acrivistine, terfenadine, promethazine, cyclizine, mizolastine, azelastine or chlorpheniramine; applied orally, topically or parenterally (for example intra-articularly).

The invention will now be illustrated by the following non-limiting Examples, in which, unless otherwise stated, the following abbreviations are used:

DMEM is tissue culture medium Dulbecco's Modified Eagles Medium PSG is a combination of penicillin, streptomycin and L-glutamine

FCS is foetal calf serum

NEAA is Non essential amino acids

THF is tetrahydrofuran

MeOH is methanol

TBME is tert-butyl methyl ether

XRPD (X-ray powder diffraction) data were measured using a Philips X-Pert MPD machine and expressed in 0-20 configuration over the scan range 2.5° to 45° 2θ with continuous scan mode. The X-rays were generated by a copper long-fine focus tube operated at 45 kV and 40 mA. The wavelengths of the copper X-rays were 1.54056 Å (K_{α1}). The data were collected on zero background holders on which ˜2 mg of the compound was placed. The holder was made from a single crystal of silicon, which had been cut along a non-diffracting plane and then polished on an optically flat finish. The X-rays incident upon this surface were negated by Bragg extinction. XRPD data are presented in the tables below, and reflection angle (°2θ) and D-spacing (Å) data (bracketed) are provided.

### EXAMPLE 1

### N-{(2R)-3-[4-(3,4-Dichlorophenoxy)piperidin-1-yl]-2-hydroxypropyl}-2,3-dihydro-2-oxo-4-(trifluoromethyl)-5-thiazolecarboxamide benzenesulfonate Form A

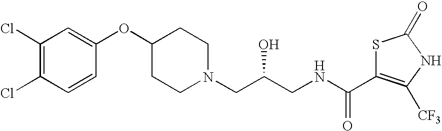

### Step 1: N-{(2R)-3-[4-(3,4-Dichlorophenoxy)piperidin-1-yl]-2-hydroxypropyl}-2,3-dihydro-2-oxo-4-(trifluoromethyl)-5-thiazolecarboxamide

A solution of ethyl 2,3-dihydro-2-oxo-4-(trifluoromethyl)-5-thiazolecarboxylic acid (85.252 g) was dissolved in thionyl chloride (500 mL) and heated at reflux overnight. The solution was cooled to room temperature, concentrated in vacuo, and residual thionyl chloride was azeotroped with toluene (2×100 mL). The residue was dissolved in dry tetrahydrofuran (250 mL) and the resulting solution was added dropwise over 3 h to a suspension of (2R)-1-amino-3-[4-(3,4-dichlorophenoxy)piperidin-1-yl]propan-2-ol (127.7 g) and triethylamine (111.5 mL) in tetrahydrofuran (1250 mL). The yellow mixture was stirred at room temperature overnight and the solution was concentrated in vacuo to leave a yellow gum (313 g). This was dissolved in water (pH 7-8) and acidified to pH 1-2 with dilute hydrochloric acid. The solid was extracted with ethyl acetate (2 L+500 mL), and sodium chloride added to the aqueous layer to salt out the remaining organics. The combined organic extractions were dried (Na₂SO₄), concentrated in vacuo and vacuum dried for 42 hours to leave a yellow foam (217 g).

MS (APCI-ve) 512/514 [M-H]⁻

¹H NMR δ (CD₃OD) 2.00-2.40 (4H, m), 3.19 (2H, m), 3.40 (2H, m), 3.20-3.60 (4H, m), 4.16-4.24 (1H, m), 4.65-4.85 (1H, m), 6.98 (1H, dd), 7.23 (1H, s), 7.43 (1H, d).

### Step 2: N-{(2R)-3-[4-(3,4-Dichlorophenoxy)piperidin-1-yl]-2-hydroxypropyl}-2,3-dihydro-2-oxo-4-(trifluoromethyl)-5-thiazolecarboxamide benzenesulfonate Form A

The yellow foam from Step 1 (above) was dissolved in methanol (400 mL) and treated with benzenesulfonic acid (63.27 g), stirred at room temperature for 2 hours, filtered and concentrated in vacuo to leave a yellow foam (297 g). This was dissolved in hot ethyl acetate (about 800 mL) and allowed to cool to room temperature. The resulting suspension was stirred at room temperature for 72 hours, filtered, washed with ethyl acetate (500 mL), air dried, and vacuum dried in an oven at 40° C. to leave the title compound as a white powder (177.5 g).

Elemental analysis: C, 44.32% (44.65); H, 3.86% (3.90); N, 6.38% (6.25); S, 9.66% (9.53).

MS (APCI-ve) 512/514 [M-H]⁻

¹H NMR δ (d₆-DMSO) 1.88-2.26 (4H, m), 3.11 (2H, m), 3.24-3.32 (2H, m), 2.86-3.46 (4H, m), 4.02-4.10 (1H, m), 4.64-4.72 (1H, m), 7.02 (1H, dd), 7.24-7.30 (4H, m), 7.50 (1H, d), 7.60-7.64 (2H, m), 8.37 (1H, s).

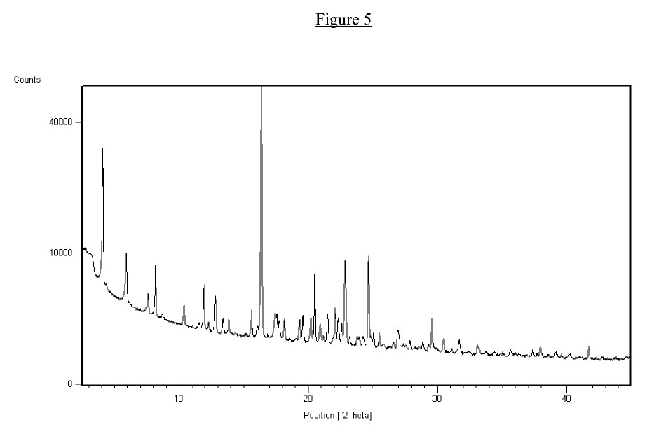

XRPD data are presented in the Table below and shown in FIG. 4

| Position | d-spacing | Rel. Int. |
|---|---|---|
| [°2θ] | [Å] | [%] |
| 4.4 | 20.21285 | 20 |
| 7.4 | 11.87237 | 18 |
| 8.7 | 10.1137 | 21 |
| 11.6 | 7.64875 | 41 |
| 12.1 | 7.33301 | 9 |
| 13.7 | 6.47789 | 4 |
| 14.3 | 6.17103 | 16 |
| 15.1 | 5.85086 | 7 |
| 15.6 | 5.67531 | 18 |
| 16.7 | 5.30891 | 2 |
| 17.5 | 5.05434 | 100 |
| 17.8 | 4.96905 | 14 |
| 18.6 | 4.77845 | 3 |
| 19 | 4.65918 | 13 |
| 19.5 | 4.55049 | 3 |
| 20.2 | 4.40054 | 26 |
| 20.4 | 4.33998 | 11 |
| 20.9 | 4.24396 | 19 |
| 21.9 | 4.04762 | 20 |
| 22.4 | 3.96495 | 5 |
| 22.8 | 3.89743 | 4 |
| 23.2 | 3.82433 | 8 |
| 23.7 | 3.75768 | 5 |
| 24 | 3.7086 | 13 |
| 24.6 | 3.60872 | 6 |
| 25.1 | 3.54588 | 16 |
| 25.3 | 3.5125 | 3 |
| 25.8 | 3.44594 | 8 |
| 26.2 | 3.39432 | 10 |
| 26.5 | 3.36615 | 4 |
| 26.9 | 3.30601 | 15 |
| 27.3 | 3.26184 | 13 |
| 27.6 | 3.22703 | 6 |
| 28.8 | 3.09631 | 3 |
| 29.6 | 3.01899 | 5 |
| 30.1 | 2.96861 | 2 |
| 30.5 | 2.92703 | 3 |
| 31.1 | 2.87438 | 5 |
| 31.5 | 2.8382 | 1 |
| 31.7 | 2.81613 | 2 |
| 32 | 2.79194 | 4 |
| 32.5 | 2.75203 | 1 |
| 33.3 | 2.68743 | 3 |
| 33.8 | 2.64968 | 2 |
| 34.4 | 2.6074 | 1 |
| 34.9 | 2.57089 | 1 |
| 35.2 | 2.55078 | 1 |
| 36.2 | 2.48138 | 3 |
| 37.1 | 2.42333 | 1 |
| 37.9 | 2.37457 | 3 |
| 38.6 | 2.32978 | 1 |
| 39.2 | 2.2991 | 2 |
| 40.2 | 2.243 | 2 |
| 40.6 | 2.21799 | 1 |
| 42.1 | 2.14502 | 1 |

### EXAMPLE 2

### N-{(2R)-3-[4-(3,4-Dichlorophenoxy)piperidin-1-yl]-2-hydroxypropyl}-2,3-dihydro-2-oxo-4-(trifluoromethyl)-5-thiazolecarboxamide benzenesulfonate Form B

### Part A: 2,3-Dihydro-2-oxo-4-(trifluoromethyl)-5-thiazolecarboxylic acid chloride

Toluene is charged to a suitable reaction vessel under an inert atmosphere (N₂ or Ar) and cooled to −6° C. under stirring. 2,3-Dihydro-2-oxo-4-(trifluoromethyl)-5-thiazolecarboxylic acid followed by Bu₄NCl are then added. The suspension is heated to 70° C. and thionyl chloride added drop-wise. The resulting suspension is heated at 75° C. for 3-5 hours. The reaction is monitored by HPLC (MeOH quench). On completion (check for end of reaction by HPLC) the now clear solution is distilled down to a third of its volume. The mixture obtained is then cooled down to 20° C. The suspension obtained is then dissolved in THF prior to use.

### Part B: (2R)-1-Amino-3-[4-(3,4-dichlorophenoxy)piperidin-1-yl]propan-2-ol

(2R)-1-Amino-3-[4-(3,4-dichlorophenoxy)piperidin-1-yl]propan-2-ol dihydrochloride and THF are charged to a second vessel and the suspension obtained stirred at 45° C. for 30 minutes. Tetramethylguanidine and THF are then added and the suspension stirred for an additional 45-60 minutes at 45° C. The suspension is then filtered and the solid residues washed with THF. The filtrate containing (2R)-1-Amino-3-[4-(3,4-dichlorophenoxy)piperidin-1-yl]propan-2-ol is recharged to the second reaction vessel, tetramethylguanidine and THF added and the solution distilled down to 7 volumes then cooled to 25° C.

### Part C: N-{(2R)-3-[4-(3,4-Dichlorophenoxy)piperidin-1-yl]-2-hydroxypropyl}-2,3-dihydro-2-oxo-4-(trifluoromethyl)-5-thiazolecarboxamide benzenesulfonate

The solution of 2,3-Dihydro-2-oxo-4-(trifluoromethyl)-5-thiazolecarboxylic acid chloride (from Part A above) is added drop-wise to (2R)-1-Amino-3-[4-(3,4-dichlorophenoxy)piperidin-1-yl]propan-2-ol (from Part B) over 1.5-2 hours at 25° C. The residues are washed in with THF. The reaction is monitored by HPLC. On completion (check for end of reaction by HPLC), the suspension is quenched by the addition of HCl 4M. The aqueous phase is removed. The organic phase is then washed with a mixture of aqueous NaCl and NaOH 10M. The aqueous phase is removed. A solution of benzenesulfonic acid in TBME is then added and the reaction mixture stirred for at least 30 minutes. The solution of N-{(2R)-3-[4-(3,4-dichlorophenoxy)piperidin-1-yl]-2-hydroxypropyl}-2,3-dihydro-2-oxo-4-(trifluoromethyl)-5-thiazolecarboxamide benzenesulfonate is then washed with an aqueous solution of NaCl, then water. The final solution is filtered and the vessel rinsed with THF.

### Part D: N-{(2R)-3-[4-(3,4-Dichlorophenoxy)piperidin-1-yl]-2-hydroxypropyl}-2,3-dihydro-2-oxo-4-(trifluoromethyl)-5-thiazolecarboxamide benzenesulfonate Form B

The filtered solution of N-{(2R)-3-[4-(3,4-Dichlorophenoxy)piperidin-1-yl]-2-hydroxypropyl}-2,3-dihydro-2-oxo-4-(trifluoromethyl)-5-thiazolecarboxamide benzenesulfonate (from Part C above) is placed in a third reaction vessel. The residues are washed in with THF. The solution is distilled down to 5 volumes. Isopropanol, 6 volumes, are then added and 6 volumes of solvents distilled out. The operation is repeated a second time. Finally, TBME, 5 volumes is added and 5 to 5.5 volumes of solvents are distilled out. The distillation's effectiveness is monitored by NMR and Karl Fischer analysis and the product precipitates out.

| Position | d-spacing | Rel. Int. |
|---|---|---|
| [°2θ] | [Å] | [%] |
| 4.1 | 21.57881 | 52 |
| 5.9 | 14.92227 | 13 |
| 7.6 | 11.644 | 4 |
| 8.2 | 10.81919 | 12 |
| 8.7 | 10.16034 | 1 |
| 10.4 | 8.52291 | 3 |
| 11.5 | 7.65614 | 1 |
| 11.9 | 7.42768 | 8 |
| 12.3 | 7.20954 | 1 |
| 12.8 | 6.91043 | 6 |
| 13.4 | 6.59583 | 2 |
| 13.8 | 6.39382 | 2 |
| 15.6 | 5.66704 | 4 |
| 16.4 | 5.40939 | 100 |
| 17.4 | 5.08314 | 4 |
| 18.2 | 4.88 | 3 |
| 19.3 | 4.58981 | 3 |
| 19.6 | 4.52793 | 3 |
| 20.2 | 4.39491 | 3 |
| 20.5 | 4.32795 | 12 |
| 20.9 | 4.24195 | 2 |
| 21.5 | 4.12833 | 4 |
| 22.1 | 4.02256 | 5 |
| 22.3 | 3.97974 | 3 |
| 22.9 | 3.88671 | 17 |
| 23.2 | 3.82836 | 1 |
| 23.9 | 3.72684 | 1 |
| 24.2 | 3.67042 | 1 |
| 24.7 | 3.60692 | 18 |
| 25.1 | 3.55012 | 1 |
| 25.5 | 3.49052 | 2 |
| 26.6 | 3.34971 | 1 |
| 27 | 3.30387 | 2 |
| 27.9 | 3.19855 | 1 |
| 28.9 | 3.09097 | 1 |
| 29.3 | 3.04471 | 1 |
| 29.6 | 3.01785 | 4 |
| 30.5 | 2.9299 | 1 |
| 31.1 | 2.872 | 0 |
| 31.7 | 2.82222 | 1 |
| 33.1 | 2.70517 | 1 |
| 34.4 | 2.6038 | 0 |
| 35.6 | 2.51838 | 0 |
| 37.4 | 2.40522 | 0 |
| 37.9 | 2.3696 | 1 |
| 39.2 | 2.29911 | 0 |
| 40.2 | 2.24023 | 0 |
| 41.7 | 2.16462 | 1 |

### EXAMPLE 3

### Characterisation of the activity of N-{(2R)-3-[4-(3,4-Dichlorophenoxy)piperidin-1-yl]-2-hydroxypropyl}-6-oxo-2-(pentafluoroethyl)-1,6-dihydropyridine-3-carboxamide (Example 35 in WO 2005/073192)

Methods

(i) Isolation of Human Articular Chondrocytes (HAC) from OA Cartilage for Histamine stimulation.

HAC are isolated from cartilage from osteoarthritic tissues removed at joint replacement. Cartilage is incubated overnight in 2 mg/ml collagenase in DMEM+10% FCS+PSG at 37° C., with shaking. The resulting cell suspension is filtered to remove undigested cartilage and the cells are centrifuged at 1200 rpm for 10 minutes. Cells are resuspended in DMEM plus 10% FCS PSG/NEAA/fungizone/gentamycin, plated at approx 5×10⁻⁶ in a T-75 and grown to confluence. Cells are subsequently plated at approx 10,000 cells per well into 96 well plates for stimulation with histamine. Triplicate wells of first passage HAC are treated with either DMEM (10% or 0% FCS) alone (control), or DMEM (10% or 0% FCS) plus histamine (dose range 10 nM up to 1 mM). Cells are incubated at 37° C. for 24 hours, the conditioned medium harvested, and assayed for PGE2, MMP or cytokine production by enzyme linked immunosorbent assay (ELISA). In addition, cells are harvested for RNA analysis, in Qiagen RLT buffer containing 1% β-mercaptoethanol.

For some experiments cells are incubated with specific antagonists for 20 minutes prior to, and in combination with, histamine stimulation.

(ii) Chemokine Stimulation of Human Cartilage Explant

Explant Preparation

Human osteoarthritic cartilage is obtained during total knee replacement surgery. Full-depth cartilage slices are obtained from both the femoral condyles and the tibial plateau and explant discs removed using a 5 mm diameter KAI sterile dermal biopsy punch. After dissection, the explants are all pooled in a Petri dish. For each condition, sixteen explants are randomly taken from the Petri dish and cultured in polypropylene 96-well plates with phenol red free Dulbecco's Modified Eagle Medium (DMEM; Gibco, Grand Island, N.Y.), 150 μg/ml gentamicin, 1.5 μg/ml fungizone and 100 units/ml penicillin, 100 μg/ml streptomycin and L-Glutamine.

After an initial 48 hour pre-stimulation, rest period, experimental reagents are added for 24, 48 or 96 hours. Explants are incubated with the appropriate concentration of compound alone, or DMSO as a control, for 20 minutes prior to addition of the chemokine. Cartilage is subsequently treated with either vehicle control (plus DMSO); chemokine (at 30-300 ng/ml) plus DMSO; or chemokine (30 ng/ml) plus the compound of Example 1. Conditioned media are collected 24, 48 or 96 hours post stimulation and stored at −20° C. prior to analysis. Cartilage explants are also collected and snap frozen in liquid nitrogen, prior to RNA extraction.

Extraction of RNA from Cartilage Explants

Each condition is composed of sixteen cartilage explant discs, which are pooled prior to snap freezing in liquid nitrogen. These pooled samples are ground to a fine powder under liquid nitrogen then resuspended in 10 ml trizol in polypropylene Oakridge tubes, and RNA extracted according to the manufacturer's protocol. The RNA pellet is resuspended in approx 500 μl RNAse and DNAse free water and then combined with 1.75 ml Qiagen RLT. RNA is further purified with Qiagen RNEasy mini columns (Qiagen Cat# 74104), as described in the manufacturer's protocol, including on column DNA digestion. The RNA obtained by these procedures is analysed using Agilent bioanalyser technology, according to the manufacturer's instructions, to determine the quantity and quality.

Gene Expression Analysis: the 96 and 48 Gene Fingerprints Using Low Density Arrays (Q-PCR)

Low-density gene arrays [quantitative polymerise chain reaction (Q-PCR) method] with either 96 or 48 genes pertinent to OA (as identified by Affymetrix microarray analysis of OA articular cartilage) are used to assess the effects of treating human diseased cartilage explants with the chemokines eotaxin-2 or RANTES, alone or in combination with the compound of Example 1. Gene expression changes were reported as fold change normalised against a standard non-affected gene: GAPDH.

(iii) In Vivo Functional Validation of Histamine-Induced Responses Murine Articular Knee Joint

It is hypothesised that histamine released by trafficking mast cells or by the de novo synthesis of histamine by articular chondrocytes induces a H1-dependant increase in COX-2 mRNA expression and elevated PGE2 synthesis in synovial fibroblasts and articular chondrocytes. To evaluate the role of histamine in pathological processes in the joint, a pharmacodynamic mouse model was developed to investigate the effect of intra-articular injection (IAI) of histamine on joint swelling using calliper measurements, and measuring PGE2 and/or 11-6 levels from synovial knee joint ravages.

Adult C57B16 mice were dosed with either vehicle or test compound 1 hour prior to intra-articular injection of histamine (5 μmol). Swelling was induced by injection of 6 μl of histamine/saline suspension into the knee joint cavity under inhalation anaesthesia with isoflurane. Joint swelling was quantified by calliper measurements taken at 1, 6, 12, 24, 36 and 48 hr. Terminal synovial fluid knee lavages were sampled and levels of the inflammatory mediator I1-6 were measured in the fluid from synovial knee joint ravages.

Results

Histamine Induced Joint Oedema

In the murine articular knee joint studies, Intra-articular injection of histamine gave a 5-fold increase in joint oedema compared to vehicle control animals. This swelling was almost completely ablated with desloratadine treatment (5.1 mg/kg).

Histamine Induced IL-6 in Joint Lavage

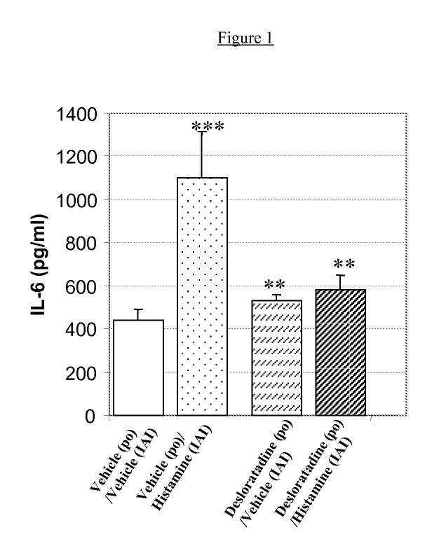

Elevated IL-6 levels have been have been observed in a number of pathological conditions, including inflammation. Induction of joint swelling by histamine injection resulted in >60% increase in release of IL-6 into the synovial fluid. All doses of desloratadine significantly inhibited IL-6 release comparable to basal levels measured for control samples (FIG. 1).

Taken together, these results show that amelioration of histamine-induced joint swelling is correlated with a reduction in synovial fluid IL-6 protein to near basal level. On this basis, the ability to limit IL-6 expression in this way can be taken to be indicative of a compound's potential activity in controlling joint swelling.

(iii) In Vitro Functional Validation of Histamine-Induced Responses in Isolated Human Articular Chondrocytes

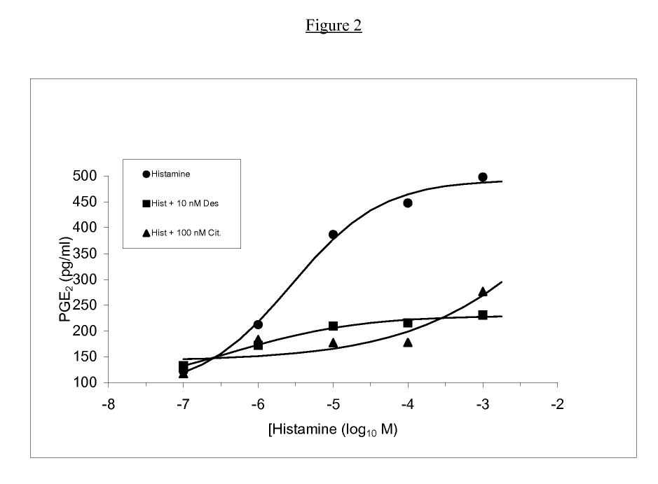

It has previously been reported that histamine induces an increase in PGE2 release by primary chondrocytes (Tetlow & Woolley 2004). In our studies we observed a histamine induced, dose dependent increase in release of PGE2 (n=6) (FIG. 2), and an increase in Cox2 mRNA expression levels in isolated human chondrocytes. These responses to histamine were inhibited by the H1R selective antagonists desloratidine (10 nM) and cetirizine (100 nM) (FIG. 2), showing that Histamine stimulated release of the pain mediator, PGE2, from isolated human chondrocytes can be abrogated by H1R antagonists.

In Vitro Functional Validation of CCR3 Mediated Responses in Human Articular Chondrocytes/Cartilage

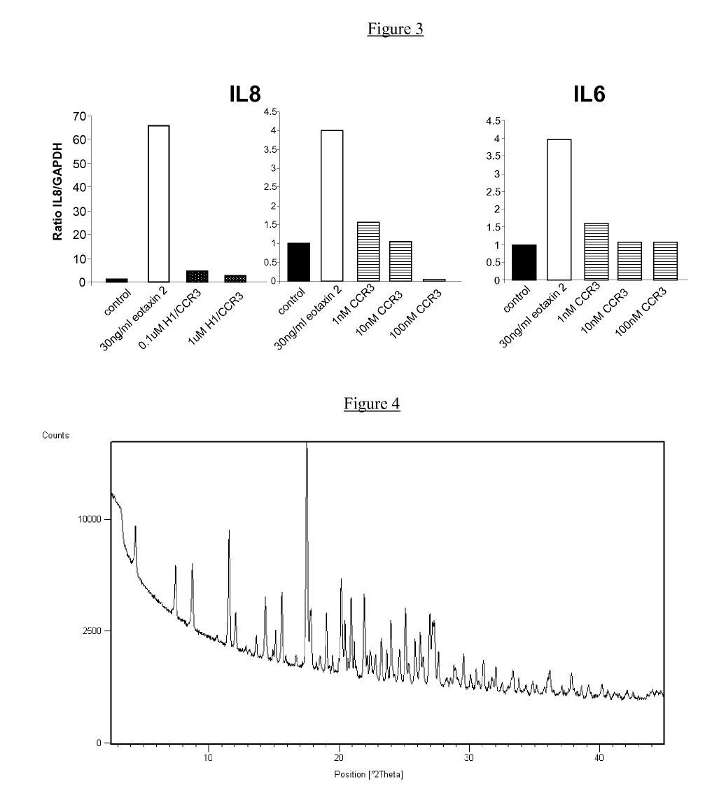

CCR3 ligands have been implicated in the pathogenesis of osteoarthritis due to their ability to modulate cartilage integrity, by increasing MMPs and stimulating loss of proteoglycans (Alaaedine et al 2001; Hsu et al 2004). The functional role of CCR3 in human cartilage explant culture by analysing the gene expression changes seen in this system in response to stimulation with ligands for CCR3 was investigated. In response to Eotaxin 2 stimulation of human explant cartilage (donor KM 014_{—}04) an increased expression of the pro-inflammatory cytokines IL-6 (14 fold) and IL-8 (11 fold) was observed. Importantly, CCR3 ligands up-regulated the metalloproteinases MMP1 (8.8 fold), MMP2 (4 fold) and MMP13 (×3.5 fold) with no concommitant change in expression of the endogenous MMP inhibitors, TIMP 1, 2 or 3. Enhanced expression of the aggrecanases, ADAMTS4 (3.6 fold) and ADAMTS 5 (6 fold) was also observed. Finally, periostin a protein postulated to be involved in the process of matrix mineralisation in OA, was modestly elevated (2 fold). These changes in gene expression were confirmed by Q-PCR analysis and ELISA. Expression of ADAMTS4, MMP13, IL-6, IL-8 and MMP2 mRNA was examined in explant cartilage from a range of donors, and a greater than 2 fold increase in mRNA expression in response to CCR3 ligands was observed in the majority of OA donors and could be inhibited by the compound of Example 1 at 3×pA2, n=3 (FIG. 3). {The y-axis of the right-hand graph in FIG. 3 (the graph headed IL6) represents the Ratio IL6/GAPDH.}

### EXAMPLE 4

A phase II, randomised, double blind, placebo controlled, parallel-group, multi-centre exploratory study to investigate the effect on clinical markers, biomarkers of collagen turnover, safety and tolerability of a compound of formula (I), or a pharmaceutically acceptable salt thereof, given orally in tablet form once daily for 4 weeks, in patients with osteoarthritis of the knee.

The objectives of the study are to evaluate the clinical effect of the compound of formula (I), or a pharmaceutically acceptable salt thereof, compared with placebo by assessing the change from baseline in WOMAC™ subscales (pain, stiffness and physical function), and the physician and patient global disease assessment. The primary collagen turnover biomarkers are planned to be CTX-II, PIIANP and uGGP (Glc-Gal-PYD).

The compound of formula (I) is, for example, the benzenesulfonate salt of N-{3-[4-(3,4-dichlorophenoxy)piperidin-1-yl]-2-hydroxypropyl}-2,3-dihydro-2-oxo-4-(trifluoromethyl)-5-thiazolecarboxamide (for example its (2R) enantiomer).
WOMAC™ index (WOMAC™ 3.1 index) is an official scoring system using a battery of 24 questions. For more information see www.womac.org/womac/index.htm.
CTX-II is a type-II collagen neo-epitope generated by MMP cleavage of type-II collagen. It is a marker of cartilage degradation.
PIIANP is the type II collagen pro-collagen alpha chain. It is a marker of cartilage synthesis.
uGGP is a maturation product of 2 hydroxyl-lysine residues from collagen C or N telopeptides with glycosylated hydroxylysine from the alpha-helix of collagen. It is a marker of synovial inflammation.
Tablet form comprises compound of formula (I), or a pharmaceutically acceptable salt thereof, microcrystalline cellulose, mannitol, sodium starch glycolate, hydroxypropylcellulose and sodium stearyl fumarate.

What is claimed:

1. A method of treating osteoarthritis or osteoarthrosis, the method comprising administering to a subject a compound of formula (I) 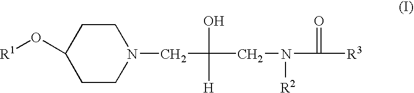
wherein:
R¹ is phenyl optionally substituted by halogen, cyano, C₁₋₄ alkyl or C₁₋₄ haloalkyl;
R² is hydrogen, C₁₋₆ alkyl or C₃₋₆ cycloalkyl; and
R³ is a group having an NH or OH that has a calculated or measured pKa of 1.0 to 8.0;
or a pharmaceutically acceptable salt.

2. The method according to claim 1 wherein R¹ is phenyl substituted with one, two or three of: halogen, cyano or C₁₋₄ alkyl

3. The method according to claim 1 wherein R² is hydrogen.

4. The method according to claim 1 wherein the acidic NH of R³ is part of a ring or is part of a substituent on an aryl or heterocyclyl ring.

5. The method according to claim 1 wherein R³ is:
2-oxo-thiazol-5-yl having a suitable electron withdrawing substituent in the 4-position;
2-oxo-oxazol-5-yl having a suitable electron withdrawing substituent in the 4-position;
1H-1,2,3-triazol-4-yl having a suitable substituent in the 5-position;
4-oxo-1H-1,4-dihydropyridin-3-yl having a suitable electron withdrawing substituent in the 2-position;
2,6-dioxo-1H-1,2,3,6-tetrahydropyrimidin-4-yl having a suitable substituent in the 3-position and optionally substituted in one or more other ring positions;
6-oxo-1H-1,6-dihydropyridin-3-yl having a suitable electron withdrawing substituent in the 2-position and/or the 5-position and optionally substituted in one or more other ring positions;
6-oxo-1H-1,6-dihydropyridin-3-yl having CH₂CO₂H on the ring nitrogen and optionally substituted in one or more other ring positions;
2H-tetrazol-5-yl;
a CO₂H, CH₂CO₂H or OCH₂CO₂H group on an optionally substituted phenyl, optionally substituted CH₂O phenyl, optionally substituted naphthyl ring; or,
an NHS(O)₂(C₁₋₄ alkyl) group on an optionally substituted aromatic heterocyclyl ring;
or, where possible, a tautomer thereof.

6. The method according to claim 1 wherein the 2-hydroxy group has the stereochemistry:

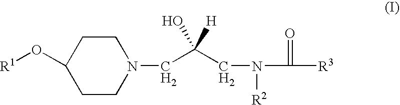

7. The method of claim 1, further comprising administering a histamine type 1 receptor antagonist.

8. The benzenesulfonate salt of N-{3-[4-(3,4-dichlorophenoxy)piperidin-1-yl]-2-hydroxypropyl}-2,3-dihydro-2-oxo-4-(trifluoromethyl)-5-thiazolecarboxamide.

9. The benzenesulfonate salt of claim 8, wherein the N-{3-[4-(3,4-dichlorophenoxy)piperidin-1-yl]-2-hydroxypropyl}-2,3-dihydro-2-oxo-4-(trifluoromethyl)-5-thiazolecarboxamide is substantially pure N-{(2R)-3-[4-(3,4-dichlorophenoxy)piperidin-1-yl]-2-hydroxypropyl}-2,3-dihydro-2-oxo-4-(trifluoromethyl)-5-thiazolecarboxamide.

10. A process for the preparation of a compound claimed in claim 8, the process comprising treating N-{3-[4-(3,4-dichlorophenoxy)piperidin-1-yl]-2-hydroxypropyl}-2,3-dihydro-2-oxo-4-(trifluoromethyl)-5-thiazolecarboxamide, in racemic form or in as the (2R) enantiomer, with benzenesulfonic acid in a suitable solvent at ambient temperature.

11. A composition comprising a compound as claimed in claim 8 in admixture with a carrier, diluent or adjuvant.

12. A method of treating asthma, rhinitis, COPD, osteoarthritis or osteoarthrosis which comprises administering to a patient a therapeutically effective amount of a compound as claimed in claim 8.

13. N-{(2R)-3-[4-(3,4-dichlorophenoxy)piperidin-1-yl]-2-hydroxypropyl}-2,3-dihydro-2-oxo-4-(trifluoromethyl)-5-thiazolecarboxamide benzenesulfonate Form A having an X-ray powder diffraction pattern containing specific peaks at: 4.4 (±0.1°), 8.7 (±0.1°), 11.6 (±0.1°), 17.5 (±0.1°), 20.2 (0.11) and 21.9 (±0.1°) 2θ.

14. A composition comprising a compound as claimed in claim 13 in admixture with a carrier, diluent or adjuvant.

15. A method of treating asthma, rhinitis, COPD, osteoarthritis or osteoarthrosis which comprises administering to a patient a therapeutically effective amount of a compound as claimed in claim 13.

16. N-{(2R)-3-[4-(3,4-dichlorophenoxy)piperidin-1-yl]-2-hydroxypropyl}-2,3-dihydro-2-oxo-4-(trifluoromethyl)-5-thiazolecarboxamide benzenesulfonate Form B having an X-ray powder diffraction pattern containing specific peaks at: 4.1 (±0.1°), 8.2 (±0.1°), 16.4 (±0.1°), 20.5 (±0.1°), 22.9 (0.11) and 24.7 (±0.1°) 2θ.

17. A composition comprising a compound as claimed in claim 16 in admixture with a carrier, diluent or adjuvant.

18. A method of treating asthma, rhinitis, COPD, osteoarthritis or osteoarthrosis which comprises administering to a patient a therapeutically effective amount of a compound as claimed in claim 16.
